(19) United States
(12) Patent Application Publication
(10) Pub. No.: US 2009/0029928 A1
(43) Pub. Date: Jan. 29, 2009

(54) PEELING PROCESS AND COMPOSITION

(75) Inventor: Odile Aubrun-Sonneville, Antony (FR)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL, Paris (FR)

(21) Appl. No.: 11/946,089

(22) Filed: Nov. 28, 2007

Related U.S. Application Data
(60) Provisional application No. 60/869,130, filed on Dec. 8, 2006.

(30) Foreign Application Priority Data
Nov. 30, 2006 (FR) 06 55198

(51) Int. Cl. A61K 31/7048; A61K 31/19; A61K 31/192; A61K 31/375

(52) U.S. Cl. 514/27; 514/557; 514/568; 514/474

(57) ABSTRACT

Process for the treatment of visible and/or tactile irregularities of human skin, by applying topically, to the skin, a composition containing at least one hydroxy acid chosen from α-hydroxy acids, β-hydroxy acids, α-keto acids, β-keto acids, and mixtures thereof, and at least one cationic polymer, leaving the composition in contact with the skin for a period of time sufficient for the composition to act, and optionally, removing the composition by rinsing. Associated compositions.

### REFERENCE TO PRIOR APPLICATIONS

This application claims priority to U.S. provisional application 60/869,130 filed Dec. 8, 2006, and to French patent application 0655198 filed Nov. 30, 2006, both incorporated herein by reference.

### FIELD OF THE INVENTION

The present invention relates to a peeling process which uses a composition containing at least one hydroxy acid and at least one cationic polymer and to the associated compositions.

Additional aspects and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Peels are a well-known means for improving the surface appearance of the skin, in particular for treating visible and/or tactile irregularities of human skin, and, for example, reducing pigmentation defects such as actinic lentigo or acne or chickenpox marks, or for smoothing out skin texture irregularities, in particular wrinkles and fine lines.

These peels have the effect of removing a part of the skin to be treated (epidermis and, possibly, superficial layer of the dermis), by means of chemical methods such as the application of compositions containing high concentrations of agents for stimulating desquamation of the skin, for instance hydroxy acids such as glycolic acid or salicylic acid, or else other active agents such as, for example, retinoic acid, resorcin, trichloroacetic acid or phenol. Thus, document U.S. Pat. No. 6,787,148 describes anhydrous compositions containing a phenol and a polyethylene glycol derivative.

Increasing concentrations of active agents, and in particular of hydroxy acids, make it possible to increase the effectiveness of products. However, such concentrations lead to considerable discomfort upon application and after application (redness, stinging, burning sensation). Thus, salicylic acid peels can give rise to cases of salicylism in the event of overdose or of sustained application.

In order to improve the tolerance of peeling compositions based on hydroxy acids, it has, for example, been proposed, in document U.S. Pat. No. 6,787,148, to combine therewith polyethylene glycols such as polyethylene glycol 1500. However, the introduction of such compounds is detrimental to the cosmetic qualities of the composition, since they induce a tacky effect and lead to poor rinsability of the composition.

### SUMMARY OF THE INVENTION

There remains therefore the need to have peeling compositions which are effective while at the same time being better tolerated and having good cosmetic properties.

The inventor has found, surprisingly, that the combination of hydroxy acid(s) and of at least one cationic polymer provide peeling compositions which are both effective and well tolerated and have good cosmetic properties.

### DETAILED DESCRIPTION OF THE PREFERRED EMBODIMENTS

There was nothing to suggest in the prior art that cationic polymers could be combined with AHAs or BHAs for performing cosmetic chemical peels that are both effective and well tolerated. EP-A-680748 describes compositions containing hydroxy acids and cationic polymers, but these compositions are used for depigmenting the skin and not for peels. Moreover, document EP-A-395282 describes an aqueous acidic solution thickened with cationic polymers, but the use of the solution as a cosmetic chemical peel is not described.

The present invention relates in a preferred embodiment to a peeling process.

A subject of the invention is therefore a process for the treatment of visible and/or tactile irregularities of human skin, comprising applying topically, to the skin, a composition comprising, in a physiologically acceptable medium, (i) at least 5% by weight of one or more hydroxy acids chosen from α-hydroxy acids, β-hydroxy acids, α-keto acids, β-keto acids, and mixtures thereof, relative to the total weight of the composition, and (ii) at least one crosslinked cationic polymer (step a). Generally, of course, the user leaves the composition in contact with the skin for a period of time sufficient for the composition to act (step b), and optionally removes the composition, for example by rinsing (step c).

The composition is preferably left in contact with the skin for an application time which is sufficient for the composition to act. This time will vary according to the concentration of hydroxy acids in the composition and to the desired effect. By way of indication, and not limitation, the composition may remain in contact with the skin or the integuments for a period of at least 5 minutes, generally between 5 minutes and 12 hours, preferably between 5 minutes and 6 hours, preferentially between 5 minutes and 30 minutes. The composition may or may not be removed at the end of this period of contact. The application may be daily or twice-daily, or weekly, etc., and may be repeated if desired for any period, such as periods of 2 weeks to 6 months, it being possible for this period to be shortened, prolonged or renewed without difficulty.

Since the composition is intended for topical application, it preferably comprises a physiologically acceptable medium. The term “physiologically acceptable medium” is intended to mean a medium compatible with keratin materials such as the skin, the lips, the nails, the scalp and/or the hair. The composition is in particular a cosmetic or dermatological composition, more particularly for use in skin peeling.

The medium is preferably an aqueous medium, i.e. it contains water, the amount of which is preferably at least 20% by weight relative to the total weight of the composition. The amount of water can range, for example, from 20% to 95% by weight, preferably from 30% to 90% by weight, and better still from 35% to 80% by weight, relative to the total weight of the composition.

The composition preferably has a pH which can range from 0.4 to 7, more preferably from 0.5 to 5, and better still from 1 to 4.

The composition according to the invention is preferably free of PP-14 butyl ether.

Hydroxy Acids

The composition according to the invention contains one or more hydroxy acids chosen from α-hydroxy acids, β-hydroxy acids, α-keto acids, β-keto acids, and mixtures thereof. According to a preferred embodiment of the invention, the hydroxy acids are chosen from α-hydroxy acids and α-keto acids, and mixtures thereof.

As α-hydroxy acids, mention may more particularly be made, without limitation, of citric acid, lactic acid, glycolic acid, tartaric acid, malic acid, mandelic acid, methyllacetic acid, glucuronic acid, pyruvic acid, phenylacetic acid, gluconic acid, galacturonic acid and mixtures thereof.

As β-hydroxy acids, mention may more particularly be made, without limitation, of salicylic acid and its derivatives, in particular its alkylated derivatives of formula (I) below or a salt of such a derivative:

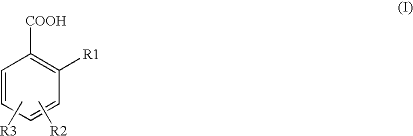

in which:
R₁ represents a hydroxyl radical or an ester of formula:

—O—CO—R₄

in which R₄ is a saturated or unsaturated aliphatic radical containing from 1 to 26 carbon atoms, and preferably from 1 to 18 carbon atoms, or an amine or thiol function optionally substituted with an alkyl radical containing from 1 to 18 carbon atoms, and preferably 1 to 12 carbon atoms,
R₂ and R₃, independently of one another, are in position 3, 4, 5 or 6 on the benzene ring and represent, independently of one another, a hydrogen atom or a radical:

—(O)ₙ—(CO)ₘ—R₅

in which n and m, independently of one another, are each an integer equal to 0 or 1; on condition that R₂ and R₃ are not simultaneously hydrogen atoms;
R₅ represents a hydrogen atom, a linear, branched or cyclized saturated aliphatic radical containing from 1 to 18 carbon atoms or an unsaturated radical containing from 3 to 18 carbon atoms, bearing one to nine conjugated or non-conjugated double bonds, it being possible for the radicals to be substituted with at least one substituent chosen from halogen atoms (fluorine, chlorine, bromine or iodine), trifluoromethyl radicals, hydroxyl in free form or esterified with an acid containing from 1 to 6 carbon atoms, or carboxyl in free form or esterified with a lower alcohol containing from 1 to 6 carbon atoms.

The salicylic acid derivative of formula (I) is preferably such that R₁ represents a hydroxyl radical, R₂ represents a hydrogen atom, R₃ is in position 5 of the benzene ring and represents a radical —CO—R₅ in which R₅ represents a saturated aliphatic radical containing from 3 to 15 carbon atoms.

According to a preferred embodiment of the invention, the salicylic acid derivative of formula (I) is chosen from 5-n-octanoylsalicylic acid, 5-n-decanoylsalicylic acid, 5-n-dodecanoylsalicylic acid, 5-n-octylsalicylic acid, 5-n-heptyloxysalicylic acid, 4-n-heptyloxysalicylic acid, 5-tert-octylsalicylic acid, 3-tert-butyl-5-methylsalicylic acid, 3-tert-butyl-6-methylsalicylic acid, 3,5-diisopropylsalicylic acid, 5-butoxysalicylic acid, 5-octyloxysalicylic acid, 5-propanoylsalicylic acid, 5-n-hexadecanoylsalicylic acid, 5-n-oleoylsalicylic acid, 5-benzoylsalicylic acid, monovalent and divalent salts thereof, and mixtures thereof. It is more particularly 5-n-octanoylsalicylic acid (INCI: Capryloyl salicylic Acid).

As α-keto acids, mention may more particularly be made, without limitation, of ascorbic acid (or vitamin C) and ascorbyl glucosides. The term “ascorbyl glucoside” is intended to mean the product of condensation of glucose, in the D form, i.e. in the form of α- or β-glucopyranose or of α- or β-furanose, or in the L form, with ascorbic acid, preferably in the L form. Mention more especially be made of L-ascorbic acid 2-O-α-D-glucopyranoside (INCI name: Ascorbyl Glucoside), available from the company Hayashibara.

The amount of hydroxy acid(s) depends on the desired objective. It is preferably at least 5% by weight and more preferably at least 10% by weight for an effective peel. It can, for example, range from 5% to 70% by weight, preferably from 10% to 70% by weight, more preferably from 10% to 60% by weight, better still from 10% to 50% by weight, and even better still from 15% to 50% by weight, relative to the total weight of the composition.

Cationic Polymer

The composition may contain at least one crosslinked cationic polymer and may contain more than one. The amount (in active material) of cationic polymer(s) may vary according to the polymer used, and it can preferably range, for example, from 0.1% to 10% by weight, more preferably from 0.15% to 7% by weight, and better still from 0.2% to 5% by weight, and even better still from 0.5% to 5% by weight, relative to the total weight of the composition.

The cationic polymers used in the composition of the invention are crosslinked and can be homopolymers or copolymers.

The crosslinked cationic copolymers or homopolymers are preferably substantially soluble in the physiologically acceptable, e.g., aqueous, medium, and they preferably comprise units resulting from the reaction between (i) an ethylenically unsaturated cationic monomer or a cationic mixture of ethylenically unsaturated monomers, and (ii) an ethylenically polyunsaturated crosslinking agent, this crosslinking agent being present in the polymer at a concentration ranging from, for example, 5 ppm to 45 ppm, preferably from 10 to 40 ppm, and even better still from 10 to 20 ppm.

These polymers can thus be obtained, for example, conventionally according to the “emulsion polymerization” technique, from the various monomers of which they are formed.

The ethylenically polyunsaturated monomers used as crosslinking agents for the preparation of these polymers are preferably chosen from the group consisting of methylenebisacrylamide, ethylene glycol di(meth)acrylate, di(meth)acrylamide, cyanomethyl acrylate, vinyloxyethyl(meth)acrylate, or metal salts thereof. Preferably, the ethylenically polyunsaturated monomer is methylenebisacrylamide.

The cationic mixture of monomers can contain nonionic monomers in addition to the cationic monomers.

The cationic monomers that can be used are in particular, without limitation, dialkylaminoalkyl acrylates and methacrylates, and more especially dialkylaminoethyl (meth)acrylates, and quaternary salts or acids thereof; dialkylaminoalkylacrylamides or dialkylaminoalkylmethacrylamides, and quaternary salts or acids thereof, such as methacrylamidopropyltrimethylammonium chloride. The alkyl groups contain, for example, from 1 to 4 carbon atoms.

The non-ionic monomers that can be used in combination with the cationic monomers are, for example, methacrylamide or acrylamide.

The cationic polymers that can be used in the invention are in particular those described in document EP-A-395 282.

The cationic polymer can also be a polymer obtained by polymerization of a mixture of monomers comprising at least one vinyl monomer substituted with an amino group, at least one hydrophobic nonionic vinyl monomer, at least one vinyl monomer comprising an alkyl chain and an oxyalkylenated chain (referred to as associative monomer) and at least one vinyl monomer comprising an oxyalkylenated chain (referred to as surfactant monomer). These are associative polymers which are described in document US-A-2004/0052746.

The vinyl monomer substituted with an amino group can in particular be a mono- or di(C₁-C₄)alkylamino-(C₁-C₈)alkyl(meth)acrylate or mono- or di(C₁-C₄)alkylamino(C₁-C₈)alkyl(meth)acrylamide, or a nitrogenous heterocyclic (meth)acrylate or (meth)acrylamide.

The hydrophobic nonionic vinyl monomer can in particular be a C₁-C₃₀ alkyl ester of acrylic acid or methacrylic acid.

In the associative monomer, the alkyl chain can in particular be a C₈-C₄₀ chain, and the associative vinyl monomer can in particular be a polyethoxylated (C₈-C₄₀)alkyl(meth)acrylate.

The vinyl monomer comprising an oxyalkylenated chain is free of alkyl chain, and it can be a compound of formulae:

CH₂═CH—O(CH₂)ₐO(C₃H₆O)_{b}(C₂H₄O)CH or

CH₂═CH CH₂O(C₃H₆O)_{d}(C₂H₄O)_{c}H

in which a is 2, 3 or 4; b is an integer ranging from 1 to 10; c is an integer ranging from 5 to 50; d is an integer ranging from 5 to 50.

As examples of cationic polymers, mention may, for example be made of the crosslinked methacryloylethyltrimethylammonium chloride homopolymer (INCI name: POLYQUARTERNIUM-37) sold as such by the company Sigma 3V under the name Synthalen CR, or sold as a dispersion in a mixture of esters at 50% (INCI name: Polyquarternium-37 (and) Propylene glycol Dicaprylate/Dicaprate (and) PPG-1 Trideceth-6) by the company Ciba under the name SALCARE SC 96; or sold as a dispersion in a mineral oil (INCI name: Polyquarternium-37 (and) Mineral Oil (and) PPG-1 Trideceth-6) by the company Ciba under the name SALCARE SC 95; the cationic ethyl acrylate/dimethylaminoethyl methacrylate copolymer as an emulsion at 20% in water (INCI name: Polyacrylate-1 Crosspolymer) sold by the company Noveon under the name Carbopol Aqua CC, and mixtures thereof.

The compositions according to the invention can be in any form, including all of the galenic forms normally used in the cosmetics and dermatological fields, in particular in the form of aqueous gels, of lotions or of emulsions (W/O or O/W or multiple). These compositions can be prepared according to the usual methods. According to a preferred embodiment of the invention, the composition is in the form of an aqueous, aqueous-alcoholic or aqueous-glycolic gel, or of an aqueous, aqueous-alcoholic or aqueous-glycolic solution.

When the composition according to the invention comprises an oily phase, in particular when it is in the form of an emulsion, the oily phase preferably contains at least one oil, in particular physiologically acceptable oil. It can also contain other fatty substances.

As oils that can be used in the composition of the invention, mention may, for example, be made of:
hydrocarbon-based oils of animal origin, such as perhydrosqualene;
hydrocarbon-based oils of plant origin, such as liquid triglycerides of fatty acids containing from 4 to 10 carbon atoms, for instance heptanoic acid or octanoic acid triglycerides, or else, for example, sunflower oil, maize oil, soybean oil, marrow oil, grapeseed oil, sesame oil, hazelnut oil, apricot oil, macadamia oil, arara oil, castor oil, avocado oil, caprylic/capric acid triglycerides such as those sold by the company Stearineries Dubois or those sold under the names Miglyol 810, 812 and 818 by the company Dynamit Nobel, jojoba oil, shea butter oil;
synthetic esters and ethers, in particular of fatty acids, such as oils of formulae R¹COOR² and R¹OR² in which R¹ represents the residue of a fatty acid containing from 8 to 29 carbon atoms, and R² represents a branched or unbranched hydrocarbon-based chain containing from 3 to 30 carbon atoms, for instance purcellin oil, isononyl isononanoate, isopropyl myristate, 2-ethylhexyl palmitate, 2-octyldodecyl stearate, 2-octyldodecyl eructate, isostearyl isostearate, isocetyl stearate; hydroxylated esters such as isostearyl lactate, octyl hydroxystearate, octyldodecyl hydroxystearate, diisostearyl malate, triisocetyl citrate, fatty alcohol heptanoates, octanoates and decanoates; polyol esters, such as propylene glycol dioctanoate, neopentyl glycol diheptanoate and diethylene glycol diisononanoate; and pentaerythrityl esters such as pentaerythrityl tetraisostearate;
linear or branched hydrocarbons of mineral or synthetic origin, such as volatile or non-volatile paraffin oils, and derivatives thereof, petroleum jelly, polydecenes, hydrogenated polyisobutene such as parleam oil;
fatty alcohols containing from 8 to 26 carbon atoms, such as cetyl alcohol, stearyl alcohol and the mixture thereof (cetylstearyl alcohol), octyldodecanol, 2-butyloctanol, 2-hexyldecanol, 2-undecylpentadecanol, oleyl alcohol or linoleyl alcohol;
partially hydrocarbon-based and/or silicone-based fluoro oils such as those described in document JP-A-2-295912;
silicone oils such as volatile or non-volatile polymethylsiloxanes (PDMSs) containing a linear or cyclic silicone chain, which are liquid or pasty at ambient temperature, in particular cyclopolydimethylsiloxanes (cyclomethicones) such as cyclohexasiloxane; polydimethylsiloxanes comprising alkyl, alkoxy or phenyl groups which are pendent or at the end of the silicone chain, which groups contain from 2 to 24 carbon atoms; phenyl silicones such as phenyl trimethicones, phenyl dimethicones, phenyltrimethylsiloxydiphenylsiloxanes, diphenyl dimethicones, diphenylmethyldiphenyltrisiloxanes, 2-phenylethyltrimethylsiloxysilicates, and polymethylphenylsiloxanes;
mixtures thereof.

The term “hydrocarbon-based oil” in the list of oils mentioned above is intended to mean any oil containing predominantly carbon and hydrogen atoms and, optionally, ester, ether, fluoro, carboxylic acid and/or alcohol groups.

The other fatty substances that may be present in the oily phase are, for example, fatty acids containing from 8 to 30 carbon atoms, for instance stearic acid, lauric acid, palmitic acid and oleic acid; waxes such as lanolin, beeswax, carnauba wax or candelilla wax, paraffin wax, lignite wax or microcrystalline waxes, ceresin or ozokerite, synthetic waxes such as polyethylene waxes, Fischer-Tropsch waxes; silicone resins such as trifluoromethyl(C₁-C₄)alkyl dimethicone and trifluoropropyl dimethicone; and silicone elastomers such as the products sold under the name “KSG” by the company Shin-Etsu, under the names “Trefil”, “BY29” or “EPSX” by the company Dow Corning or under the name “Gransil” by the company Grant Industries.

These fatty substances can be chosen in a varied manner by those skilled in the art in order to prepare a composition having the desired properties of, for example, consistency or texture.

When the composition is in the form of an emulsion, it is preferably an oil-in-water (O/W) emulsion. The emulsions generally contain at least one emulsifier chosen in particular from amphoteric, anionic, cationic or nonionic emulsifiers, used alone or as a mixture. They are preferably nonionic emulsifiers. These emulsifiers are chosen from those conventionally used in the cosmetics field.

It is also possible to prepare emulsions without emulsifying surfactants, or containing less than 0.5% thereof, with respect to the total weight of the composition, using appropriate compounds, for example polymers having emulsifying properties, such as the polymers sold under the names Carbopol 1342 and Pemulen by the company Noveon; or optionally crosslinked and/or neutralized polymers and copolymers of 2-acrylamido-2-methylpropanesulphonic acid, such as the poly-(2-acrylamido-2-methylpropanesulphonic acid) sold by the company Clariant under the name “Hostacerin AMPS” (INCI name: ammonium polyacryldimethyltauramide) or such as the emulsion polymer sold under the name Sepigel 305 by the company Seppic (INCI name: polyacrylamide/C13-C14 isoparaffin/laureth-7); particles of ionic or nonionic polymers, more particularly particles of anionic polymer, such as in particular isophthalic acid or sulphoisophthalic acid polymers, and in particular phthalate/sulphoisophthalate/glycol copolymers (for example, diethylene glycol/phthalate/isophthalate/1,4-cyclohexanedimethanol copolymers (INCI name: diglycol/CHDM/isophthalates/SIP copolymer) sold under the names Eastman AQ polymer (AQ35S, AQ38S, AQ55S, AQ48 Ultra) by the company Eastman Chemical.

It is also possible to prepare emulsions without emulsifiers, stabilized with silicone particles or particles of metal oxide such as TiO₂ or the like, which may or may not be coated.

In a known manner, the composition of the invention may also contain adjuvants that are normal in the cosmetics or dermatological field, such as hydrophilic or lipophilic gelling agents, hydrophilic or lipophilic active agents, preserving agents (for example, phenoxyethanol and parabens), antioxidants, solvents, fragrances, fillers, bactericides, odour absorbers, dyestuffs, pH modifiers (acid or base or buffer). The amounts of these various adjuvants are those conventionally used in the field under consideration, and for example from 0.01% to 20% of the total weight of the composition. Depending on their nature, these adjuvants can be introduced into the oily phase or into the aqueous phase, or solubilized in the surfactants.

The composition according to the invention may be free of surfactants, but it may optionally contain one or more surfactants chosen from nonionic, anionic, cationic, amphoteric or zwitterionic surfactants, and mixtures thereof.

The nonionic surfactants can be chosen from surfactants comprising a group chosen, for example, from polyalkylene glycol groups, such as in particular polyethylene glycol or polypropylene glycol groups; polyglycerol groups; sugar groups (glucose, maltose, sorbitol, ethoxylated sorbitan); and mixtures thereof.

As nonionic surfactants, mention may, for example, be made of:
alkyl polyglycosides, and in particular alkyl polyglucosides (APGs) having an alkyl group containing from 6 to 16 carbon atoms (C₆-C₁₆ alkyl polyglucosides), and preferably 8 to 16 carbon atoms, for instance the decylglycoside (alkyl-C₉/C₁₁-polyglucoside (1.4)) such as the product sold under the name Mydol 10 by the company Kao Chemicals, the product sold under the name Plantaren 2000 UP or Plantacare 2000 UP by the company Cognis, and the product sold under the name Oramix NS 10 by the company Seppic; caprylyl/capryl glucoside, for instance the product sold under the name Oramix CG 110 by the company Seppic; lauryl glucoside, for instance the products sold under the names Plantaren 1200 N and Plantacare 1200 by the company Cognis; and cocoglucoside, for instance the product sold under the name Plantacare 818/UP by the company Cognis;
esters of polyethylene glycol and of acids comprising at least one alkyl chain ranging from C₆ to C₁₆, preferably from C₆ to C₁₄, such as polyethylene glycol (8 EO) myristate, for instance the product sold by the company Gattefosse under the name Mirlene;
derivatives of polyethylene glycol and of mono-, di- and triglycerides of an acid comprising at least one alkyl chain ranging from C₆ to C₁₆, preferably from C₆ to C₁₄, and having at least two ethylene oxide groups, and preferably from 6 to 8 ethylene oxide groups, such as mono-, di- and triglycerides of caprylic acid and of capric acid, for instance that comprising 6 ethylene oxide groups (INCI name: PEG-6 caprylic/capric glycerides), sold under the name Softigen 767 by the company Sasol, that comprising 8 ethylene oxide groups (INCI name: PEG-8 caprylic/capric glycerides), sold under the name L.A.S. by the company Gattefosse, and that comprising 7 ethylene oxide groups, sold under the name Cetiol HE 810 by the company Cognis (INCI name: PEG-7 caprylic/capric glycerides); the oxyethylenated (20 EO) glyceryl monolaurate sold under the name Tagat L 2 by the company Degussa-Goldschmidt;
oxyethylenated derivatives of sorbitan esters of an acid comprising at least one alkyl chain ranging from C₆ to C₁₆, preferably from C₆ to C₁₄, such as the PEG-10 sorbitan laurate sold under the name Liposorb L10 by the company Lipo Chemicals;
sugar esters comprising at least one C₆ to C₁₆ alkyl chain, such as the mixture of sucrose laurate and sucrose dilaurate, sold by the company Mitsubishi Chemical under the name Surfhope SE Cosme C-1216;
esters of polyglycerol and of an acid comprising at least one C₆ to C₁₆ alkyl chain, such as the polyglycerol monolaurate (10 mol of glyceryl) (INCI name: polyglyceryl-10 laurate) sold by the company Sakamoto Yakuhin under the name S Face L-1001;
polyglyceryl ethers, such as the polyglycerol-3 hydroxylauryl ether produced by Chimex under the name Chimexane NF;
ethers of polyethylene glycol and of fatty alcohols comprising a C₈ to C₃₀ alkyl chain, such as ethers of cetyl alcohol, of stearyl alcohol, of cetearyl alcohol or of lauryl alcohol,
and mixtures thereof.

As anionic surfactants, mention may, for example, be made of:
alkyl sulphates, alkyl ether sulphates and their salts, in particular their sodium salts, for instance sodium lauryl ether sulphate such as the product sold under the name Texapon AOS 225 UP by the company Cognis;
monoalkyl and dialkyl esters of phosphoric acid, and their salts, for instance sodium mono- and dilauryl phosphate, potassium mono- and dilauryl phosphate, triethanolamine mono- and dilauryl phosphate, sodium mono- and dimyristoyl phosphate, potassium mono- and dimyristoyl phosphate, diethanolamine mono- and dimyristoyl phosphate, triethanolamine mono- and dimyristoyl phosphate;
amino acid derivatives, in particular the alkali metal salts of amino acids, such as:
   acyl sarcosinates, such as the sodium lauroyl sarcosinate sold under the name Sarkosyl NL 97® by the company Ciba or sold under the name Oramix L 30® by the company Seppic, the sodium myristoyl sarcosinate sold under the name Nikkol Sarcosinate MN® by the company Nikkol, the sodium palmitoyl sarcosinate sold under the name Nikkol Sarcosinate PN® by the company Nikkol;
   acyl alaninates, such as the sodium N-lauroyl-N-methyl aminopropionate sold under the name Sodium Nikkol Alaninate LN 30® by the company Nikkol, or sold under the name Alanone ALE® by the company Kawaken, the N-lauroyl-N-methyl alanine triethanolamine sold under the name Alanone Alta® by the company Kawaken;
   acyl glutamates, such as the triethanolamine monococoyl glutamate sold under the name Acylglutamate CT-12® by the company Ajinomoto, the triethanolamine lauroyl glutamate sold under the name Acylglutamate LT-12® by the company Ajinomoto;
   acyl glycinates, such as the sodium N-cocoyl glycinate sold under the names Amilite GCS-12® and Amilite GCK 12 by the company Ajinomoto;
alkyl ether carboxylates, in particular those of formula:

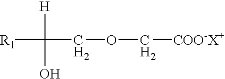

in which:

R1 denotes more particularly a linear or branched, saturated or unsaturated alkyl radical containing from 8 to 16 carbon atoms,

X denotes hydrogen or a mineral or organic cation such as those derived from an alkali metal (for example, Na⁺ or K⁺), NH₄⁺, ammoniums derived from basic amino acids such as lysine, arginine, sarcosine, ornithine or citrulline, or alternatively amino alcohols such as monoethanolamine, diethanolamine, triethanolamine, glucamine, N-methylglucamine or 3-amino-1,2-propanediol. Preferred 2-hydroxy alkyl ether carboxylic acids are compounds of formula (I) in which R1 denotes more particularly a mixture of C₈-C₁₆ radicals, in particular derived from coconut. Among the surfactants of formula (I), mention may in particular be made of the product sold under the name Beaulight Shaa by the company Sanyo;
and mixtures thereof.

The cationic surfactants that can be used according to the present invention may in particular, but without limitation, be primary, secondary or tertiary amine salts comprising a C₆ to C₁₆ alkyl chain, which are optionally polyoxyalkylenated; quaternary ammonium salts; imidazoline derivatives or amine oxides that are cationic in nature.

In the quaternary ammonium salts, the anion is preferably a halide (chloride, bromide or iodide) or an alkyl sulphate, more particularly a methyl sulphate. It is, however, possible to use quaternary ammonium salts in which the anion is a methanesulphonate, a phosphate, a nitrate, a tosylate, an anion derived from an organic acid, such as acetate or lactate, or any other anion compatible with the ammonium comprising an ester function. The anion is even more particularly chloride or methyl sulphate.

Among the quaternary ammonium salts, mention may in particular be made of:
tetraalkylammonium chlorides such as, for example, dialkyldimethylammonium chloride or alkyltrimethylammonium chloride, in which the alkyl radical contains approximately from 6 to 16 carbon atoms, for instance the dodecyltrimethylammonium chloride sold under the name Arquad 12-50 by the company Akzo Nobel, or the cetyltrimethylammonium chloride sold under the name Dehyquart A or by the company Cognis;
quaternary ammonium salts containing at least one ester function, for example the dicocoylethylhydroxyethylmethylammonium methosulphate sold under the name Dehyquart L 80 by the company Cognis;
quaternary ammonium salts containing a sugar unit, for example a glucose, fructose or sucrose unit, for instance the butyldimoniumhydroxypropyl laurylglucoside chloride (INCI name: butyldimoniumhydroxypropyl laurylglucoside chloride) sold under the name Colonial SugaQuat TM-1212 by the company Colonial Chemical Inc, the lauryl methyl gluceth-10 hydroxypropyldimonium chloride (INCI name: lauryl methyl gluceth-10 hydroxypropyldimonium chloride) sold under the name Glucquat 125 by the company Noveon.

The amphoteric and zwitterionic surfactants can be chosen, for example, from betaine derivatives, and in particular alkylated derivatives of betaine, alkylamidopropylbetaines, alkylamphoacetates, hydroxysultaines, and mixtures thereof.

As betaine derivatives, mention may in particular be made of alkylbetaines comprising a C₆-C₁₆, and more particularly C₆-C₁₄, alkyl group, and their oxyethylenated derivatives of these alkylbetaines, for example cocobetaine, for instance the product sold under the name Dehyton AB-30® by the company Cognis, laurylbetaine, for instance the product sold under the name Genagen KB® by the company Clariant, oxyethylenated (10 EO) laurylbetaine, for instance the product sold under the name Laurylether (10 EO) Betaine® by the company Shin Nihon Rica, or oxyethylenated (10 EO) stearylbetaine, for instance the product sold under the name Stearylether (10 EO) Betaine® by the company Shin Nihon Rica.

As alkylamidopropylbetaines, mention may, for example, be made of (C₆-C₁₆)alkylamidopropylbetaines such as the cocoamidopropylbetaine sold, for example, under the name Velvetex BK 35® by the company Cognis, or else the undecyleneamidopropyl betaine sold, for example, under the name Amphoram U® by the company Ceca.

As alkylamphoacetates, mention may, for example, be made of (C₆-C₁₆)alkylamphoacetates, such as N-disodium N-cocoyl-N-carboxymethoxyethyl-N-carboxymethylethylenediamine (INCI name: disodium cocoamphodiacetate), for instance the product sold under the name Miranol C2M Concentrate NP® by the company Rhodia Chimie, and N-sodium N-cocoyl-N-hydroxyethyl-N-carboxymethylethylenediamine (INCI name: sodium cocoamphoacetate).

The amount of surfactants (in terms of active material) can range, for example, from 0.1% to 70% by weight, preferably from 0.5% to 60% by weight, better still from 1% to 50% by weight, and even better still from 1% to 30% by weight, relative to the total weight of the composition.

Of course, those skilled in the art will take care to select the possible additive(s) to be added to the composition according to the invention, and the amounts thereof, in such a way that the advantageous properties intrinsically associated with the composition in accordance with the invention are not, or not substantially, impaired by the addition envisaged.

According to a specific embodiment of the invention, the composition contains at least one hydrophilic, i.e. water-soluble or water-dispersible, polymer.

As hydrophilic polymers, mention may in particular be made of:
cellulose-based derivatives (carboxymethylcellulose, hydroxyethyl cellulose, hydroxypropylmethylcellulose);
natural gums such as xanthan gum, guar gum, carob gum or carrageenans;
polycarboxyvinyl polymers of the carbomer type, such as those sold by the company Goodrich under the names Carbopol 940, 951 and 980, or by the company 3V-Sigma under the name Synthalen K or Synthalen L;
acrylic copolymers such as the acrylate/alkylacrylate copolymers sold under the name Pemulen by the company Goodrich;
polyacrylamides and acrylamide copolymers, indicated above, such as the product sold under the name Sepigel 305 by the company Seppic, the product sold under the name Hostacerin Amps by the company Clariant or the copolymers sold under the name Aristoflex by the company Clariant.

The amount of hydrophilic polymer(s) can range, for example, from 0.01% to 5% by weight, preferably from 0.05% to 5% by weight, and better still from 0.1% to 3% by weight, relative to the total weight of the composition.

Moreover, the composition can also contain at least one water-soluble hydroxylated compound chosen from C₂-C₆, and preferably C₂-C₄, monohydric alcohols such as ethanol and isopropanol, and polyols containing from 1 to 3 carbon atoms, such as glycerol, propylene glycol, butylene glycol, dipropylene glycol, isopropylene glycol; and mixtures thereof. The amount of hydroxylated compound(s) can range, for example, from 0.1% to 70% by weight, better still from 1% to 65% by weight, and even better still from 1 to 60% by weight, relative to the total weight of the composition.

When the composition is aqueous-alcoholic, it contains water and at least one monohydric alcohol, and when the composition is aqueous-glycolic, it contains water and at least one polyol. It can of course contain both alcohols and polyols.

The composition can also contain any appropriate active agent other than the hydroxy acids mentioned above, such as, for example, urea and its hydroxylated derivatives, for instance the N-(2-hydroxyethyl)urea sold under the name hydrovance by the company National Starch: hyaluronic acid; hydrating polymers such as acrylic polymers comprising a phosphorylcholine group, such as:
the poly-2-(methacryloyloxyethyl)phosphorylcholine at 40% in a water/butanediol mixture (5% of butanediol) sold under the name Lipidure HM by the company Nippon Oils and Fats (INCI name: polyphosphorylcholine glycol acrylate (and) butylene glycol);
the 2-(methacryloyloxyethyl)phosphorylcholine/butyl methacrylate (90/10) copolymer at 5% in solution in water, sold under the name Lipidure PMB by the company Nippon Oils and Fats (INCI name: Polyquarternium-51);
the 2-(methacryloyloxyethyl)phosphorylcholine/2-hydroxy-3-methacryloyloxypropyltrimethylammonium chloride copolymer at 5% in solution in water, sold under the name Lipidure-C by the company Nippon Oils and Fats;
the 2-(methacryloyloxyethyl)phosphorylcholine/butyl methacrylate/sodium methacrylate terpolymer at 5% in solution in water, sold under the name Lipidure-A by the company Nippon Oils and Fats;
the 2-(methacryloyloxyethyl)phosphorylcholine/stearyl methacrylate copolymers sold under the names Lipidure-S, Lipidure-NR and Lipidure-NA by the company Nippon Oils and Fats (INCI name: Polyquarternium-61).

The composition may also contain other active agents such as vitamins, for instance vitamins A, C, E, B3, B5 and K, and derivatives thereof, in particular esters thereof, and sequestering agents such as EDTA.

As fillers, the composition of the invention may contain, for example, mineral particles such as clays, silicas, metal oxides such as titanium dioxide or zinc oxide, or mica, and/or organic fillers such as particles of polyamide (nylon) and in particular those sold under the name Orgasol by the company Atochem; lattices; polyethylene powders; acrylic copolymer-based microspheres, such as those made of ethylene glycol dimethacrylate/lauryl methacrylate copolymer, sold by the company Dow Corning under the name Polytrap; poly(methyl methacrylate) microspheres, sold under the name Microsphere M-100 by the company Matsumoto or under the name Covabead LH85 by the company Wackherr; ethylene/acrylate copolymer powders, such as those sold under the name Flobeads by the company Sumitomo Seika Chemicals; expanded powders such as hollow microspheres, and in particular the microspheres formed from a vinylidene chloride/acrylonitrile/methacrylate terpolymer and sold under the name Expancel by the company Kemanord Plast; powders of natural organic materials, such as starch powders, in particular crosslinked or noncrosslinked maize, wheat or rice starch powders, such as the powders of starch crosslinked with octenylsuccinate anhydride, sold under the name Dry-Flo by the company National Starch; silicone resin microbeads such as those sold under the name Tospearl by the company Toshiba Silicone, in particular Tospearl 240; and mixtures thereof. The amount of filler(s) can range, for example, from 0.05% to 20% by weight, and better still from 0.1% to 10% by weight, relative to the total weight of the composition.

As indicated above, this composition is intended to be used in a peeling process aimed at reducing the visible and/or tactile irregularities of the skin, and in particular at reducing wrinkles and fine lines and/or pigmentary marks and/or scars, in particular acne marks, and/or at unblocking the pores of the skin and giving the skin a more radiant look. The composition can therefore be applied in particular to the face and/or the neck and/or the neck and shoulders and/or the hands and/or the back.

In order to optimize its effects, the peeling process according to the invention preferably comprises additional steps of preparing the skin for the peeling and/or of caring for the skin after peeling, using compositions containing smaller amounts of active agents than the peeling composition described above.

The implementation of the above preliminary step also makes it possible to screen for any possible allergy to the active agents and to improve the effectiveness and homogeneity of the peel.

Thus, according to a specific embodiment, the process according to the invention comprises, in addition to the steps mentioned above:
a preliminary step of applying, to the skin, a composition comprising, in a physiologically acceptable medium, either from 0.1% to 10% by weight of one or more cationic polymers, relative to the total weight of the composition, or 3% to 10% by weight of one or more hydroxy acids, relative to the total weight of the composition, or 3% to 10% by weight of a mixture of one or more cationic polymers and of one or more hydroxy acids, relative to the total weight of the composition, before the implementation of step (a), and/or
an additional step of applying, to the skin, a composition containing, in a physiologically acceptable medium, either from 10% to 50% by weight of one or more cationic polymers, relative to the total weight of the composition, or from 10% to 50% of one or more hydroxy acids, relative to the total weight of the composition, or from 10% to 50% of a mixture of one or more cationic polymers and of one or more hydroxy acids, relative to the total weight of the composition, after the implementation of step (c).

The compositions used in these preliminary and additional steps can be applied in the morning and in the evening, for example, optionally in combination with a composition intended to protect the skin against the effects of UV radiation. The pre-treatment composition can be applied for one to four weeks, and the post-treatment composition for one day to eight weeks, for example.

The process according to the invention, including the optional preliminary and additional steps, can be carried out just once or repeated for example up to five times if necessary, etc., the peel sessions preferably being separated by one to eight weeks.

The invention will now be illustrated by means of the following nonlimiting examples. In these examples, the amounts are indicated as percentage by weight, and they represent amounts of active material. As appropriate, the names indicated are the chemical names or the INCI names.

### Examples 1 to 5 According to the Invention

| | Exam- | Exam- | Exam- | Exam- | Exam- |
|---|---|---|---|---|---|
| | ple 1 | ple 2 | ple 3 | ple 4 | ple 5 |
| Polyacrylate-1 | 2 | 2 | — | — | 3 |
| crosspolymer (1) | | | | | |
| Polyquaternium- | | | 2 | 2 | — |
| 37 (and) | | | | | |
| mineral oil | | | | | |
| (and) PPG-1 | | | | | |
| trideceth-6 (2) | | | | | |
| Glycolic acid | 30 | 50 | 20 | 50 | — |
| Lactic acid | — | — | — | — | 30 |
| Water | qs 100 | qs 100 | qs 100 | qs 100 | qs 100 |
| pH | 1.2 | 0.7 | 1.5 | 0.46 | not |
| | | | | | measured |

(1) Carbopol Aqua CC from Noveon, which is at 30% with respect to active material. In order to have 2% of active material, 6.67% of starting material are used, and in order to have 3% of active material, 10% of starting material is used.

(2) Salcare SC95 from Ciba, which is at 50% with respect to active material. In order to have 2% of active material, 4% of starting material is used.

Protocol: The cationic polymer is dispersed in water, optionally by heating slightly, and then the AHAs are added with stirring.

The composition obtained can be used for the skin peel, and it is as effective as a composition containing an equivalent amount of AHA, while at the same time being less aggressive, as shown by the tests described below.

In Vitro Tolerance of the Peeling Products

The tolerance of the products was evaluated in vitro on reconstructed epidermis (EpiSkin^{SM}).

The protocol used (EpiPeel) makes it possible to set up a strategy for evaluating highly concentrated active agents in a simple formulation. This protocol uses the complementarity of two tests carried out in vitro on reconstructed epidermides: predictive test for corrosion (validated in Europe as test for replacing the animal model, TG 431), and predictive test for the irritant potential of chemical products. EpiPeel makes it possible to define a “standard” graduation of the effects over 5 levels of activity. It is currently aimed at evaluating the skin tolerance of formulated or nonformulated products at high concentrations, the recommended contact times of which are short and carried out under control.

EpiPeel Predictive Model

| | Level 1 | Level 2 | Level 3 | Level 4 | Level 5 |
|---|---|---|---|---|---|
| Corrosion | R35 | R34 | R34 | NC | NC |
| EU | | | | | |
| Corrosion | Class | Class | Class | NC | NC |
| UN | I | II | III | | |
| Tolerance | | | | Irritant | Non |
| | | | | | irritant |

NC: noncorrosive
Predictive model for corrosion (OECD guideline TG 431): R35/classI: severe corrosive
% viability <35% after 3 min of contact
R34/class II: corrosive
% viability ≧35% after 3 min of contact and <35% after 1 hour of contact
R34/class III: corrosive
% viability ≧35% after 1 hour of contact and <35% after 4 hours of contact
UN: United Nations classification
EU: European Union classification

The corrosive potential of Example 1 was found to be R34/class III, whereas that of a 30% glycolic acid solution at the same pH was found to be R34/class II; this solution is therefore more corrosive than Example 1 according to the invention.

In Vitro Peel Effectiveness Test

An in vitro test was carried out in order to determine the effectiveness of Example 1 according to the invention compared with a composition with no cationic polymer, comprising 30% of glycolic acid and 70% of water. The number of corneocytes released after application of the composition according to the invention was approximately 80/μl for the two supports. The in vitro peel effectiveness of said supports is therefore equivalent.

Consequently, the composition according to the invention has the advantage of being better tolerated for identical effectiveness.

The above written description of the invention provides a manner and process of making and using it such that any person skilled in this art is enabled to make and use the same, this enablement being provided in particular for the subject matter of the appended claims, which make up a part of the original description and including a process for the cosmetic treatment of visible and/or tactile irregularities of human skin, comprising the steps consisting in:

(a) applying topically, to the skin, a composition comprising, in a physiologically acceptable medium, (i) at least 5% by weight of one or more hydroxy acids chosen from α-hydroxy acids, β-hydroxy acids, α-keto acids, β-keto acids, and mixtures thereof, relative to the total weight of the composition, (ii) at least one crosslinked cationic polymer,

(b) leaving the composition in contact with the skin for a period of time sufficient for the composition to act, and

(c) optionally removing the composition by rinsing.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted. Phrases such as “mention may be made,” etc. preface examples of materials that can be used and do not limit the invention to the specific materials, etc., listed.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

The invention method and composition is preferably used by subjects desirous of the benefits noted herein, subjects “in need of” these benefits. Such subjects include those that are or may be suffering from signs of ageing of the skin generally, from pigmentation defects such as actinic lentigo or acne or chickenpox marks, from skin texture irregularities, in particular wrinkles and fine lines, from age-related hollowing of the face and/or cheeks, from age-related changes to the contour of the eyes, etc., such as by self diagnosis or cosmetician or medical diagnosis, or are at recognized and appreciated risk of developing such conditions and who use the invention methods and compositions to combat these effects. In this regard, the invention process can be viewed as one for delaying the onset of the appearance of, and/or for reducing signs of, ageing of the skin, through the peeling process.

Naturally, one using the invention as disclosed will use an amount of the invention composition effective to reduce the signs of ageing. Such amount is inclusive of an amount of the compositions described herein at the disclosed concentrations of active ingredients sufficient to cover the area of the skin being treated in a single application, and of course includes that amount applied upon repeated application, for example on a daily basis over a course of days, weeks, etc. In a preferred embodiment the invention process includes multiple applications of the invention composition to the area(s) of skin in need of attention.

1. A process for the cosmetic treatment of visible and/or tactile irregularities of human skin, comprising:
(a) applying topically, to the skin, a composition comprising, in a physiologically acceptable medium, (i) at least 5% by weight of one or more hydroxy acids chosen from α-hydroxy acids, β-hydroxy acids, α-keto acids, β-keto acids, and mixtures thereof, relative to the total weight of the composition, and (ii) at least one crosslinked cationic polymer,
(b) leaving the composition in contact with the skin for a period of time sufficient for the composition to act, and
(c) optionally removing the composition by rinsing.

2. The process according to claim 1, wherein the composition comprises at least 20% by weight of water relative to the total weight of the composition.

3. The process according to claim 1, comprising at least one α-hydroxy acid chosen from citric acid, lactic acid, glycolic acid, tartaric acid, malic acid, mandelic acid, methyllacetic acid, glucuronic acid, pyruvic acid, phenylacetic acid, gluconic acid, galacturonic acid, and mixtures thereof.

4. The process according to claim 1, comprising at least one β-hydroxy acid chosen from salicylic acid and its alkylated derivatives of formula (I) below or a salt of such a derivative: 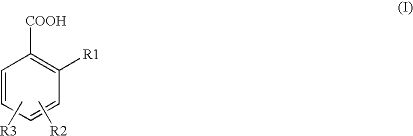
in which:
R₁ represents a hydroxyl radical or an ester of formula:
—O—CO—R₄
in which R₄ is a saturated or unsaturated aliphatic radical containing from 1 to 26 carbon atoms, or an amine or thiol function optionally substituted with an alkyl radical containing from 1 to 18 carbon atoms,
R₂ and R₃, independently of one another, are in position 3, 4, 5 or 6 on the benzene ring and represent, independently of one another, a hydrogen atom or a radical:
—(O)ₙ—(CO)ₘ—R₅
in which n and m, independently of one another, are each an integer equal to 0 or 1; on condition that R₂ and R₃ are not simultaneously hydrogen atoms;
R₅ represents a hydrogen atom, a linear, branched or cyclized saturated aliphatic radical containing from 1 to 18 carbon atoms or an unsaturated radical containing from 3 to 18 carbon atoms, bearing one to nine conjugated or non-conjugated double bonds, it being possible for the radicals to be substituted with at least one substituent chosen from halogen atoms, trifluoromethyl radicals, hydroxyl in free form or esterified with an acid containing from 1 to 6 carbon atoms, or carboxyl in free form or esterified with a lower alcohol containing from 1 to 6 carbon atoms.

5. The process according to claim 1, comprising at least one α-keto acid chosen from ascorbic acid and ascorbyl glucosides.

6. The process according to claim 1, wherein the amount of hydroxy acid(s) ranges from 5% to 70% by weight relative to the total weight of the composition.

7. The process according to claim 1, wherein the amount of cationic polymer(s) ranges from 0.1% to 10% by weight relative to the total weight of the composition.

8. The process according to claim 1, wherein the cationic polymer is a crosslinked cationic polymer consisting of units resulting from the reaction between (i) an ethylenically unsaturated cationic monomer or a cationic mixture of ethylenically unsaturated monomers, and (ii) an ethylenically polyunsaturated crosslinking agent.

9. The process according to claim 1, wherein the cationic polymer is obtained by polymerization of a mixture of monomers comprising at least one vinyl monomer substituted with an amino group, at least one hydrophobic nonionic vinyl monomer, at least one vinyl monomer comprising an alkyl chain and an oxyalkylenated chain, and at least one vinyl monomer comprising an oxyalkylenated chain.

10. The process according to claim 1, wherein the cationic polymer is chosen from crosslinked methacryloylethyltrimethylammonium chloride homopolymer and cationic ethyl acrylate/dimethylaminoethyl methacrylate copolymer, and mixtures thereof.

11. The process according to claim 1, wherein the composition is left in contact with the skin for a period of between 5 minutes and 12 hours.

12. The process according to claim 1, further comprising a preliminary step of applying, to the skin, a primary composition comprising, in a physiologically acceptable medium, either from 0.1% to 10% by weight of one or more cationic polymers, relative to the total weight of the primary composition, or from 3% to 10% of one or more hydroxy acids, relative to the total weight of the primary composition, or from 3% to 10% of a mixture of one or more cationic polymers and of one or more hydroxy acids, relative to the total weight of the primary composition, before the implementation of (a).

13. The process according to claim 1, further comprising an additional step of applying, to the skin, a secondary composition comprising, in a physiologically acceptable medium, either from 10% to 50% by weight of one or more cationic polymers, relative to the total weight of the secondary composition, or from 10% to 50% by weight of one or more hydroxy acids, relative to the total weight of the secondary composition, or from 10% to 50% of a mixture of one or more cationic polymers and of one or more hydroxy acids, relative to the total weight of the secondary composition, after the implementation of (c).

14. The process according to claim 1, wherein said composition is applied to the skin of a person in need of reducing wrinkles and fine lines and/or pigmentary marks and/or acne marks, and/or to unblock the pores of the skin.

15. The process according to claim 1, wherein the composition is applied to the face and/or the neck and/or the neck and shoulders and/or the hands and/or the back.

16. The process according to claim 1, wherein the composition is left in contact with the skin for a period of between 5 minutes and 12 hours, wherein the amount of hydroxy acid(s) ranges from 5% to 70% by weight relative to the total weight of the composition, wherein the amount of cationic polymer(s) ranges from 0.1% to 10% by weight relative to the total weight of the composition, and wherein said composition is applied to the skin of a person in need of reducing wrinkles and fine lines and/or pigmentary marks and/or acne marks, and/or to unblock the pores of the skin.
